(19) United States
(12) Patent Application Publication
(10) Pub. No.: US 2024/0242832 A1
(43) Pub. Date: Jul. 18, 2024

(54) LATE-ONSET NEONATAL SEPSIS PREDICTIONS

(71) Applicant: KONINKLIJKE PHILIPS N.V., EINDHOVEN (NL)

(72) Inventors: GREGORY BOVERMAN, READING, MA (US); SHRUTI GOPAL VIJ, CAMBRIDGE, MA (US); KRISTEN TGAVALEKOS, CAMBRIDGE, MA (US)

(21) Appl. No.: 18/289,375

(22) PCT Filed: May 5, 2022
(86) PCT No.: PCT/EP2022/062225

§ 371 Date: Nov. 3, 2023

Related U.S. Application Data
(60) Provisional application No. 63/184,880, filed on May 6, 2021.

(51) Int. Cl. G16H 50/20; A61B 5/00; G16H 10/40; G16H 10/60; G16H 15/00; G16H 50/50

(52) U.S. Cl. CPC G16H 50/20; A61B 5/412; G16H 10/40; G16H 10/60; G16H 15/00; G16H 50/50; A61B 2503/045

(57) ABSTRACT

A controller (150) includes a processor (152) that executes instructions to apply trained artificial intelligence to new measurements of vital signs of a neonatal patient and new laboratory results from laboratory tests of the neonatal patient, to changes between the new measurements of vital signs of the neonatal patient and previous measurements of vital signs of the neonatal patient, and to changes between the new laboratory results from laboratory tests of the neonatal patient and previous laboratory results from laboratory tests of the neonatal patient. The controller (150) is configured to compute, based on applying the trained artificial intelligence after retrieving the new measurements of vital signs of the neonatal patient and the new laboratory results from laboratory tests of the neonatal patient, a determination in advance indicating whether the neonatal patient will be diagnosable with late-onset neonatal sepsis.

### BACKGROUND

Late-onset neonatal sepsis is variably differentiated from early-onset neonatal sepsis by how long an infant is in a medical facility (e.g., a hospital's newborn intensive care unit or NICU) before onset of sepsis. Sepsis can be extraordinarily dangerous for infants, and especially infants born at the earliest survivable gestational ages. It is now possible for extremely premature infants born at even 22 or 23 weeks gestational age (GA) to survive. While the rate of neonatal sepsis for extremely premature infants may have decreased in recent years, among these infants, the rate of neonatal sepsis is as high as 36%. Overall, the rate of mortality from neonatal sepsis is 16% but it is as high as 54% for infants 22-24 weeks gestational age.

Recently, risk score calculators have been introduced for estimating the risk of early-onset sepsis (EOS) for late-preterm infants born at or over 35 weeks gestational age, primarily using maternal risk factors such as the length of time of rupture of membranes or maternal Group-B Streptococcus infection status. However, there is no existing tool or algorithm for assessing the risk in more premature infants born at fewer than 35 weeks gestational age. The lack of clinical standards for assessing neonatal sepsis risk has led to significant practice variation.

Many neonates born at 34 or fewer weeks gestational age with early-onset neonatal sepsis are treated with empiric antibiotics. However, this is not the case for late-onset hospital-acquired neonatal sepsis. Extremely premature neonates will tend to spend several months in the hospital and it would not be practical or effective to treat them continuously with antibiotics. In fact, excessive use of antibiotics is associated with increased mortality in very low birth-weight neonates.

Neonates who incur late-onset sepsis are likely to be quite premature because extremely premature neonates tend to stay much longer in the NICU than infants born at full term.

Neonatal sepsis relates to various specific biomarkers, such as the concentration of bands and segs (specific immune cells), platelets, and other cell types. Research shows that these biomarkers are associated with a high risk of sepsis, but in the context of a classification algorithm these same biomarkers do not show great utility because the prevalence of these abnormal laboratory results is quite low. In addition, low birth weight and low gestational age are associated with an elevated risk of late onset sepsis, but these risk factors are well-known to clinicians and do not add much clinical value. There has also been recent research into predictive models for neonatal sepsis. A large-scale meta-analysis found that in general the models had modest predictive accuracy and that the clinical parameters with statistically significant (p-value<0.05) pooled associations were lethargy, pallor, lipid infusion, and total parenteral nutrition (TPN). One prediction model is the NOSEP mode, and this model was trained and validated with a relatively small cohort of international data, achieving an AUC (area under the curve) of 0.8.

Another clinical decision support tool for detecting neonatal sepsis is the HERO score, which is based on heart-rate variability, a promising biomarker associated with infection in neonates. However, this biomarker alone achieves only an AUC of 0.73 and even combined with abnormal test results the AUC was 0.82. The current version of the HERO score does not incorporate laboratory results or other information related to clinical context, which is a significant limitation.

### SUMMARY

A controller includes a memory that stores instructions and a processor that executes the instructions. When executed by the processor, the instructions cause the controller to obtain and store measurements of vital signs of a neonatal patient and laboratory results from laboratory tests of the neonatal patient: query for new measurements of vital signs of the neonatal patient and new laboratory results from laboratory tests of the neonatal patient and retrieve the new measurements of vital signs of the neonatal patient and the new laboratory results from laboratory tests of the neonatal patient: and append the new measurements of vital signs of the neonatal patient and the new laboratory results from laboratory tests of the neonatal patient to previous measurements of vital signs of the neonatal patient and previous laboratory results from laboratory tests of the neonatal patient. The controller is configured to apply trained artificial intelligence to the new measurements of vital signs of the neonatal patient and the new laboratory results from laboratory tests of the neonatal patient, to changes between the new measurements of vital signs of the neonatal patient and the previous measurements of vital signs of the neonatal patient, and to changes between the new laboratory results from laboratory tests of the neonatal patient and the previous laboratory results from laboratory tests of the neonatal patient, and compute, based on applying the trained artificial intelligence after retrieving the new measurements of vital signs of the neonatal patient and the new laboratory results from laboratory tests of the neonatal patient, a determination in advance indicating whether the neonatal patient will be diagnosable with late-onset neonatal sepsis.

A method includes obtaining and storing measurements of vital signs of a neonatal patient and laboratory results from laboratory tests of the neonatal patient: querying for new measurements of vital signs of the neonatal patient and new laboratory results from laboratory tests of the neonatal patient and retrieving the new measurements of vital signs of the neonatal patient and the new laboratory results from laboratory tests of the neonatal patient: and appending the new measurements of vital signs of the neonatal patient and the new laboratory results from laboratory tests of the neonatal patient to previous measurements of vital signs of the neonatal patient and previous laboratory results from laboratory tests of the neonatal patient. The method also includes applying trained artificial intelligence to the new measurements of vital signs of the neonatal patient and the new laboratory results from laboratory tests of the neonatal patient, to changes between the new measurements of vital signs of the neonatal patient and the previous measurements of vital signs of the neonatal patient, and to changes between the new laboratory results from laboratory tests of the neonatal patient and the previous laboratory results from laboratory tests of the neonatal patient, and computing, based on applying the trained artificial intelligence after retrieving the new measurements of vital signs of the neonatal patient and the new laboratory results from laboratory tests of the neonatal patient, a determination in advance indicating whether the neonatal patient will likely be diagnosable with late-onset neonatal sepsis.

A system includes a memory that stores instructions, and a processor that executes the instructions. When executed by the processor, the instructions cause the system to obtain and store measurements of vital signs of a neonatal patient and laboratory results from laboratory tests of the neonatal patient: query for new measurements of vital signs of the neonatal patient and new laboratory results from laboratory tests of the neonatal patient and retrieve the new measurements of vital signs of the neonatal patient and the new laboratory results from laboratory tests of the neonatal patient: and append the new measurements of vital signs of the neonatal patient and the new laboratory results from laboratory tests of the neonatal patient to previous measurements of vital signs of the neonatal patient and previous laboratory results from laboratory tests of the neonatal patient. The system is configured to apply trained artificial intelligence to the new measurements of vital signs of the neonatal patient and the new laboratory results from laboratory tests of the neonatal patient, to changes between the new measurements of vital signs of the neonatal patient and the previous measurements of vital signs of the neonatal patient, and to changes between the new laboratory results from laboratory tests of the neonatal patient and the previous laboratory results from laboratory tests of the neonatal patient, and compute, based on applying the trained artificial intelligence after retrieving the new measurements of vital signs of the neonatal patient and the new laboratory results from laboratory tests of the neonatal patient, a determination in advance indicating whether the neonatal patient will be diagnosable with late-onset neonatal sepsis.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a system for late-onset neonatal sepsis predictions, in accordance with a representative embodiment.

FIG. 2 illustrates a method for late-onset neonatal sepsis predictions, in accordance with a representative embodiment.

FIG. 3 illustrates Shapley feature importance of a plurality of features used for late-onset neonatal sepsis predictions, in accordance with a representative embodiment.

FIG. 4 illustrates a method for late-onset neonatal sepsis predictions, in accordance with a representative embodiment.

FIG. 5 illustrates a ROC curve for a random forest classifier for late-onset neonatal sepsis predictions, in accordance with a representative embodiment.

FIG. 6 illustrates a computer system, on which a method for late-onset neonatal sepsis predictions is implemented, in accordance with another representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As described herein, a model has been built to forecast detection of late-onset neonatal sepsis six to twenty four hours in advance from vital signs and laboratory data stored in an electronic medical record (EMR) system. In developing the model, both the values of the vital sign and laboratory test parameters and their changes over time in specific time windows preceding the current value are used. The parameters may be encoded as feature vectors, which may be given as input to a classification engine, such as a random forest model. Additionally, inputs to the model may include features such as whether a given parameter was measured and when it was last measured. The output of the model is the probability of receiving a positive microbiology culture result or the neonate receiving antibiotics for 96 hours (or longer) beginning six to twenty four hours in the future. The resulting rapid recognition of late-onset neonatal sepsis may in turn lead to improved clinical outcomes.

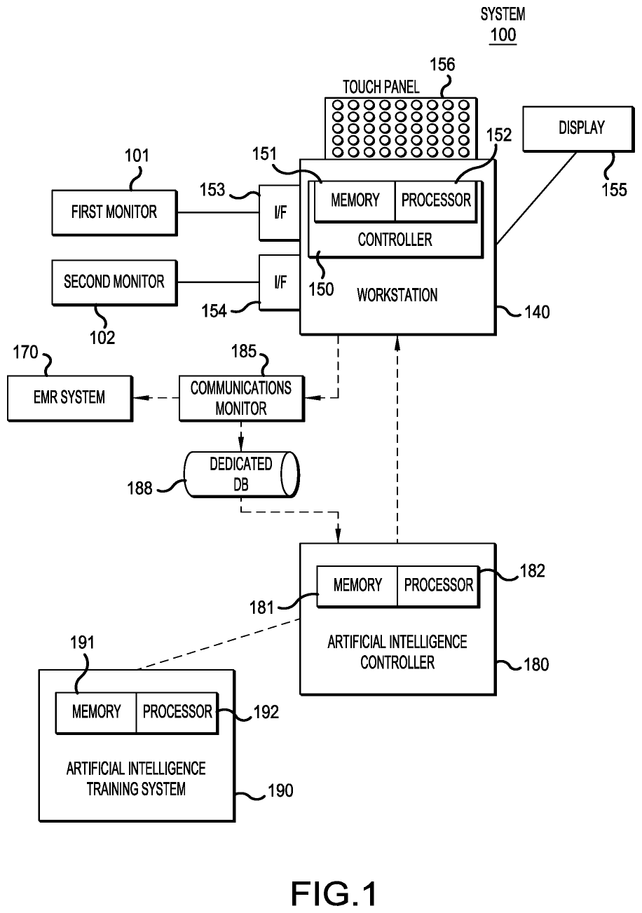

FIG. 1 illustrates a system for late-onset neonatal sepsis predictions, in accordance with a representative embodiment.

The system 100 in FIG. 1 is a system for late-onset neonatal sepsis predictions and includes components that may be provided together or that may be distributed. The system 100 includes a first monitor 101, a second monitor 102, a workstation 140, an EMR system 170, an artificial intelligence controller 180, a communications monitor 185, and a dedicated database 188. An artificial intelligence training system 190 also is shown, and provides trained artificial intelligence for implementation by the artificial intelligence controller 180.

The workstation 140 includes a controller 150, a first interface 153, a second interface 154, a display 155 and a touch panel 156. The controller 150 includes a memory 151 that stores instructions and a processor 152 that executes the instructions. The first interface 153 interfaces the workstation 140 to the first monitor 101. The second interface 154 interfaces the workstation 140 to the second monitor. The display 155 may be local to the main body of the workstation 140, and may be connected to the main body of the workstation 140 via a local wired interface such as an Ethernet cable or via a local wireless interface such as a Wi-Fi connection.

The first interface 153 and the second interface 154 are input interfaces that are representative of interfaces present in the system 100. Interfaces present in the system 100 may include ports, disk drives, wireless antennas, or other types of receiver circuitry. The interfaces present in the system 100 may further connect user interfaces, such as a mouse, a keyboard, a microphone, a video camera, the display 155, the touch panel 156, the artificial intelligence controller 180 and/or the communications monitor 185 to the workstation 140.

The display 155 may be a monitor such as a computer monitor, a display on a mobile device, a television, an electronic whiteboard, or another screen configured to display electronic imagery. The display 155 displays visualizations such as medical imagery, medical data, medical signals or information based on medical signals generated by the first monitor 101 and by the second monitor 102, and other types of information appropriate for display in a medical setting such as a hospital room. The display 155 may also include one or more interface(s) such as those noted above that may connect other elements or components to the workstation 140, as well as a touch screen that enables direct input via touch.

The touch panel 156 accepts touch input from users, such as input to a soft keyboard, and/or input from a mouse or keyboard.

The EMR system 170 stores electronic medical records for patients. The EMR system 170 may include one or more electronic medical record databases which store electronic medical records for patients. The electronic medical records stored by the EMR system may include comprehensive medical data obtained from numerous different sources for each patient, such as in a hospital environment. In some embodiments, the EMR system 170 may store electronic medical records from multiple different medical facilities such as for a hospital system with multiple hospitals.

The artificial intelligence controller 180 includes a memory 181 that stores instructions and a processor 182 that executes the instructions. The artificial intelligence controller 180 applies trained artificial intelligence in the manner described herein.

The communications monitor 185 intercepts measurements of vital signs of the neonatal patient and laboratory results from laboratory tests of the neonatal patient by monitoring transmissions sent to the EMR system 170. The communications monitor 185 monitors communications to the EMR system 170. The communications monitor 185 intercepts, copies and forwards a copy of specific types of communications for storage in the dedicated database 188.

The dedicated database 188 stores data intercepted by the communications monitor 185, and appends new measurements to previous measurements for each patient being monitored. The stored data is fed to the artificial intelligence controller 180 for repeated applications of artificial intelligence to predict whether late-onset neonatal sepsis is likely. The dedicated database 188 stores data by recording the measurements of vital signs and laboratory measurements for specific patients being monitored for late-onset neonatal sepsis.

As described above, an artificial intelligence controller 180 may provide a digital processing system, the dedicated database 188 provides a storage repository for measurement data, and the artificial intelligence controller 180 interfaces the dedicated database 188 over electronic interfaces to query the dedicated database periodically for new measurements. The measurements are combined in various ways as input features to a classification algorithm, for example a random forest algorithm which is based on a number of pooled decision trees, each of which is created by resampling the data randomly, with replacement.

Given the measurements, the algorithm computes a binary decision indicating its best guess as to whether the neonate is infected with late-onset sepsis. Additionally, the probability of infection at any given time can be computed, in case a hard decision is not desirable. Finally, for each decision, the contribution of each feature to the final score can be computed, giving its relative importance.

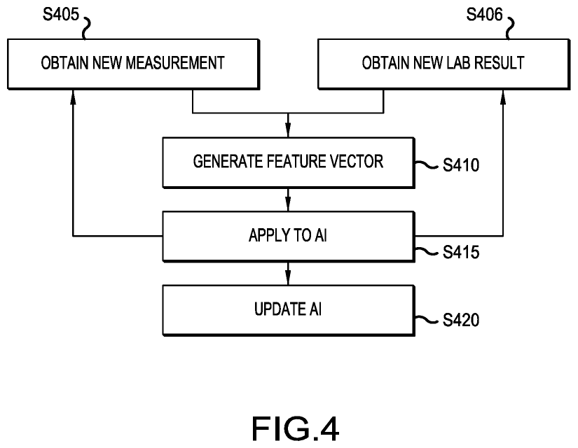

The artificial intelligence training system 190 trains artificial intelligence that is provided for implementation by the artificial intelligence controller 180. The artificial intelligence training system 190 may implement a method described with respect to FIG. 4 below, and acts by training the trained artificial intelligence implemented by the artificial intelligence controller 180.

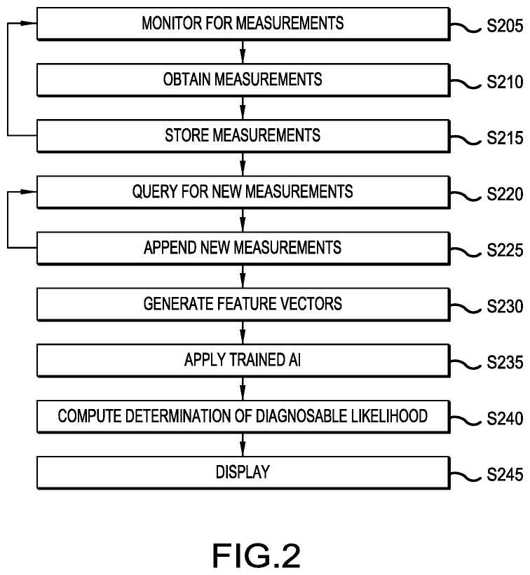

FIG. 2 illustrates a method for late-onset neonatal sepsis predictions, in accordance with a representative embodiment.

At S205, the method of FIG. 2 includes monitoring for measurements. The monitoring for measurements at S205 may be performed by the communications monitor 185, and may be monitoring for specific types of measurements or other data as described herein.

At S210, the method of FIG. 2 includes obtaining measurements. The measurements obtained at S210 are obtained by the communications monitor 185 and provided to the dedicated database 188. The measurements may be vital signs of a neonatal patient and/or laboratory results from laboratory tests of the neonatal patient.

At S215, the method of FIG. 2 includes storing the measurements. The measurements stored at S215 may be stored in the dedicated database 188. After storing the measurements at S215, the monitoring at S205 resumes while the method of FIG. 2 proceeds to S220.

At S220, the method of FIG. 2 includes querying for new measurements. The query for new measurements at S220 may be a query from the artificial intelligence controller 180 to the dedicated database 188 to query for whether new measurements have been stored at S215. The query at S220 may be periodic or based on a specific trigger, such as if/when the communications monitor 185 notifies the artificial intelligence controller 180 that a specific type of measurement has been obtained and stored. The query for new measurements may be a query for new measurements of vital signs of the neonatal patient and new laboratory results from laboratory tests of the neonatal patient.

At S225, the method of FIG. 2 includes appending new measurements. The new measurements of vital signs of the neonatal patient and the new laboratory results from laboratory tests of the neonatal patient may be retrieved from the dedicated database 188 and appended to previous measurements of vital signs of the neonatal patient and previous laboratory results from laboratory tests of the neonatal patient stored in the artificial intelligence controller 180. The process at S225 returns to S220, and the method of FIG. 2 includes repeatedly querying the dedicated database 188 for new measurements.

At S230, the method of FIG. 2 includes generating feature vectors. Feature vectors used during operations in the method of FIG. 2 may be similar to feature vectors used to train the trained artificial intelligence s described later for the method of FIG. 4. The feature vectors are generated for application as input to a machine-learning algorithm used as the trained artificial intelligence. The feature vectors may include the most recent measurement for each parameter, the time since the most recent measurement, and time-series features such as the mean, median, and variance of the measurement in the preceding thirty six hours. The feature vector may also include the time since the last measurement of a parameter was made and if the parameter was ever measured.

At S235, the method of FIG. 2 includes applying trained artificial intelligence. The trained artificial intelligence is applied to the new measurements of vital signs of the neonatal patient and the new laboratory results from laboratory tests of the neonatal patient, to changes between the new measurements of vital signs of the neonatal patient and the previous measurements of vital signs of the neonatal patient, and to changes between the new laboratory results from laboratory tests of the neonatal patient and the previous laboratory results from laboratory tests of the neonatal patient. The new measurements may be point-in-time measurements, whereas the changes between measurements may reflect changed between measurements over time. The method of FIG. 2 may include applying the trained artificial intelligence each time a query at S220 identifies a new measurement of vital signs or a new laboratory result from a laboratory test of the neonatal patient. The method of FIG. 2 may apply the trained artificial intelligence to each new measurement until reaching a determination indicating that the neonatal patient will likely be infected with late-onset sepsis.

At S240, the method of FIG. 2 includes computing a determination of a diagnosable likelihood. The determination computed at S240 is a determination computed in advance indicating whether the neonatal patient will be diagnosable with late-onset neonatal sepsis in a future period from six hours to twenty four hours in advance. The determination may be based on training described with respect to FIG. 4, and the training may set a specific minimum time such as twelve hours in advance. Therefore, the determination at S240 may be a determination of a probability of late-onset neonatal sepsis being incurred at least a specific time in the future such as at least twelve hours in the future.

The computation at S240 may be based on a particular combination of vital signs, laboratory results, and time-series measurements, which can be extracted from the EMR system 170. The risk score from the computation at S240 may provide clinicians an objective and useful tool assisting in the difficult clinical decision, such as whether to give antibiotics to a neonate on any given day. A risk score may be a score on a scale such as 1 to 100, a letter score on a scale such as from A to F or from A to Z. or any other type of scoring mechanism which will clearly convey to a clinician that late-onset neonatal sepsis is being predicted now when it was not being predicted before.

At S245, the method of FIG. 2 includes displaying results, such as the determination of the diagnosable likelihood. The displaying at S245 is representative of how an alert, an alarm, or another type of warning or informational output may be presented. In some embodiments, an alert or alarm may be sent via text messages, emails, lighting indicators. Additionally, although not shown in FIG. 2, results may require confirmation, such as from one or more clinicians. Confirmation may be provided, for example, by turning off an alert or alarm, or replying to a message or email.

As shown for the system in FIG. 1 and described with respect to the method of FIG. 2, nurses and other clinicians may manually enter measurements and observations into an electronic medical record (EMR) such as the EMR system 170 in FIG. 1. In parallel, other hospital data systems, such as a laboratory system and patient physiological monitors, may also provide data for entry into the EMR system 170. The communications monitor 185 listens to the transmissions being sent to the EMR system 170 and collects data for the prediction algorithm implemented by the artificial intelligence controller 180 into the customized database implemented by the dedicated database 188. The communications monitor 185 may be provided insofar as the EMR system may not be designed to accept repeated queries on a real-time basis. At regular time intervals, the workstation 140 or the artificial intelligence controller 180 may query the dedicated database 188 for each patient, computes the feature vector needed for input into the algorithm applied by the artificial intelligence controller 180, and deploy the trained machine learning model, which computes the probability of infection. The probability, along with other relevant clinical information, such as trends in the parameters deemed to be most significant, may then be shown on an output clinical display such as the display 155, utilized by clinicians in their decision-making.

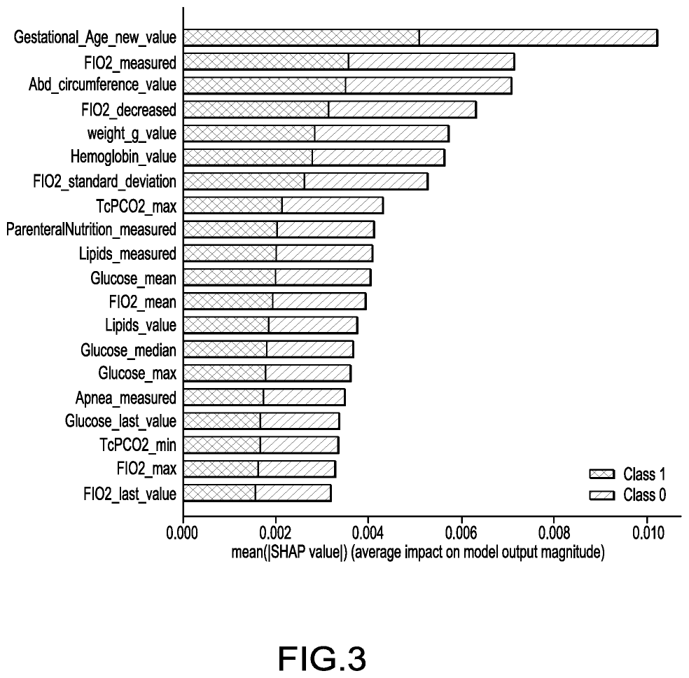

FIG. 3 illustrates Shapley feature importance of a plurality of features used for late-onset neonatal sepsis predictions, in accordance with a representative embodiment.

The Python SHAP package may be used to estimate the Shapley feature importance of each feature used in the classification algorithm, with the results shown in FIG. 3. The most important feature shown in FIG. 3 is the corrected gestational age, which is gestational age+weeks since birth. The second most important feature shown in FIG. 3 is whether FIO2 was measured in the thirty six hours preceding the decision time, which is equivalent to asking whether the neonate was on supplemental oxygen at any point during that period of time. Other important features in FIG. 3 are the abdominal circumference and changes in FIO2, for example the standard deviation of this parameter over the past thirty six hours. Therefore, not only parameter values but also their changes over time may be considered in assessing a neonate's sepsis status.

FIG. 4 illustrates a method for late-onset neonatal sepsis predictions, in accordance with a representative embodiment.

FIG. 4 shows a methodology for training a machine learning algorithm for the detection of late-onset hospital-acquired sepsis. The methodology of FIG. 4 may be performed by the artificial intelligence training system 190 in FIG. 1, and trained artificial intelligence may be provided from the artificial intelligence training system 190 as or to the artificial intelligence controller 180. The machine-learning algorithm may be, for example, a random forest or a neural network.

The methodology of FIG. 4 may start by obtaining a large database of retrospective data. A data set for extremely premature infants may be a large multi-institution data set.

The training may involve correlating measurements with one of three neonate states: 1.) infection. 2.) pre-infection, and 3.) not infected. The neonate state may be inferred by looking at whether the neonate is currently being given antibiotics or whether a blood culture has been ordered. The time of suspicion may be taken to be the earlier of the two times: i.e., the earlier of the time that antibiotics were given or the time that a blood culture was ordered. If a blood culture was ordered and the result is not known in the retrospective database, then all subsequent data for this neonate may be discarded, as at this time the infection state is not known. For the methodology in FIG. 4, infection state may be defined as either a positive blood culture result or a period of administration of antibiotics 96 hours in duration or longer. The period of administration of antibiotics may be based on clinical expert opinion, and the 96 hours in duration may be the duration in which antibiotics are administered. The methodology of FIG. 4 is not limited to the details noted herein, as other clinically based criteria defining the presence of infection are also possible. For non-infected and infected neonates, a “control” data point may also be considered where the neonate was known not to be infected, based on either the absence of antibiotics, the absence of a culture, or a negative culture result.

For each case of infection in the methodology of FIG. 4, the early infection detection time may be taken to be twelve hours beforehand. That is, with the database of retrospective data, the training in FIG. 4 “learns” how to forecast detection of late-onset neonatal sepsis twelve hours in advance. The method of FIG. 4 begins at S405 by obtaining a new measurement and/or at S406 by obtaining a new lab result. Each new measurement and/or new lab result from the database may be treated as a data point to be used as the basis for generating feature vectors for application to the artificial intelligence.

At S410, the method of FIG. 4 includes generating a feature vector for the new measurement from S405 or new lab result from S406. Given the early-infection and control data points, feature vectors are generated for application as input to the machine-learning algorithm. The feature vectors may comprise the most recent measurement for each parameter, the time since the most recent measurement, and time-series features such as the mean, median, and variance of the measurement in the preceding thirty six hours. The feature vector may also include the time since the last measurement of a parameter was made and if the parameter was ever measured.

At S415, the feature vector is applied to the artificial intelligence. Each feature vector may be associated with a binary outcome, i.e., whether the infant in the next twelve hours has an infection confirmed by a bacterial culture or the infant begins a course of antibiotics with duration greater than or equal to ninety six hours. Given the pairs of feature vectors and outcomes: (xi=1:N,yi=1:N), the machine-learning algorithm is used to generalize to unseen data vectors x, predicting the unknown outcome. This is accomplished by partitioning the data into three sets: a training set, used to train the classification algorithm, a validation set used to estimate the optimal hyperparameters of the classification algorithm, and a test set with a purpose to assess the performance of the algorithm on unseen data. After the feature vector is applied at S415, the method of FIG. 4 includes updating the artificial intelligence at S420, and also returning to S405 and/or S406 to obtain new measurements or new lab results.

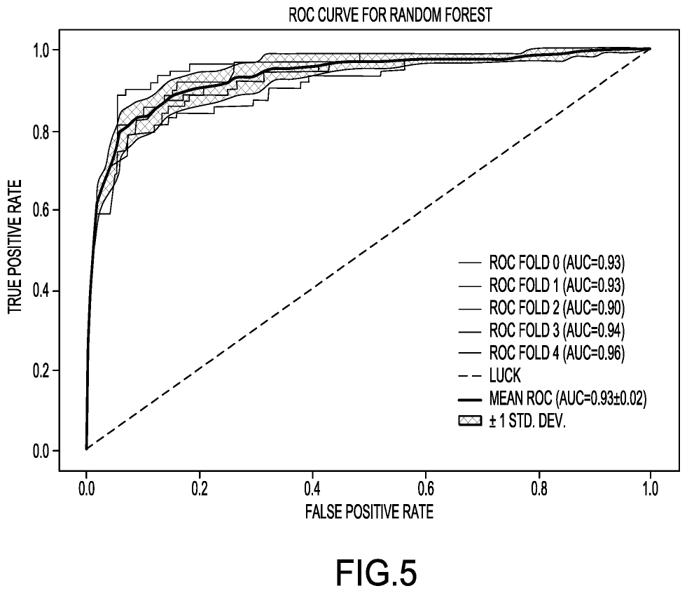

FIG. 5 illustrates a ROC curve for a random forest classifier for late-onset neonatal sepsis predictions, in accordance with a representative embodiment.

The model trained using the training in FIG. 4 achieves a mean area-under-the curve (AUC) of 0.93 as shown in FIG. 5, as assessed using five-fold cross-validation. The results shown in FIG. 3 reflect training based on a large cohort of data from a number of hospitals in the United States.

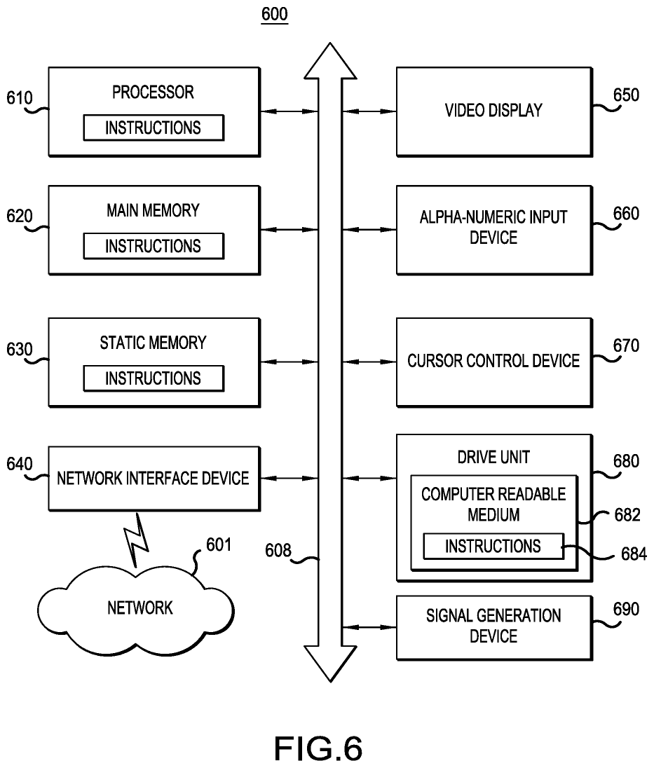

FIG. 6 illustrates a computer system, on which a method for late-onset neonatal sepsis predictions is implemented, in accordance with another representative embodiment.

The computer system 600 of FIG. 6 shows a complete set of components for a communications device or a computer device. However, a “controller” as described herein may be implemented with less than the set of components of FIG. 6, such as by a memory and processor combination. The computer system 600 may include some or all elements of one or more component apparatuses in a system for late-onset neonatal sepsis predictions herein, although any such apparatus may not necessarily include one or more of the elements described for the computer system 600 and may include other elements not described.

Referring to FIG. 6, the computer system 600 includes a set of software instructions that can be executed to cause the computer system 600 to perform any of the methods or computer-based functions disclosed herein. The computer system 600 may operate as a standalone device or may be connected, for example, using a network 601, to other computer systems or peripheral devices. In embodiments, a computer system 600 performs logical processing based on digital signals received via an analog-to-digital converter.

In a networked deployment, the computer system 600 operates in the capacity of a server or as a client user computer in a server-client user network environment, or as a peer computer system in a peer-to-peer (or distributed) network environment. The computer system 600 can also be implemented as or incorporated into various devices, such as the workstation 140 in FIG. 1, a stationary computer, a mobile computer, a personal computer (PC), a laptop computer, a tablet computer, or any other machine capable of executing a set of software instructions (sequential or otherwise) that specify actions to be taken by that machine. The computer system 600 can be incorporated as or in a device that in turn is in an integrated system that includes additional devices. In an embodiment, the computer system 600 can be implemented using electronic devices that provide voice, video or data communication. Further, while the computer system 600 is illustrated in the singular, the term “system” shall also be taken to include any collection of systems or sub-systems that individually or jointly execute a set, or multiple sets, of software instructions to perform one or more computer functions.

As illustrated in FIG. 6, the computer system 600 includes a processor 610. The processor 610 may be considered a representative example of the processor 152 of the controller 150 in FIG. 1 and executes instructions to implement some or all aspects of methods and processes described herein. The processor 610 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 610 is an article of manufacture and/or a machine component. The processor 610 is configured to execute software instructions to perform functions as described in the various embodiments herein. The processor 610 may be a general-purpose processor or may be part of an application specific integrated circuit (ASIC). The processor 610 may also be a microprocessor, a microcomputer, a processor chip, a controller, a microcontroller, a digital signal processor (DSP), a state machine, or a programmable logic device. The processor 610 may also be a logical circuit, including a programmable gate array (PGA), such as a field programmable gate array (FPGA), or another type of circuit that includes discrete gate and/or transistor logic. The processor 610 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 600 further includes a main memory 620 and a static memory 630, where memories in the computer system 600 communicate with each other and the processor 610 via a bus 608. Either or both of the main memory 620 and the static memory 630 may be considered representative examples of the memory 151 of the controller 150 in FIG. 1, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 620 and the static memory 630 are articles of manufacture and/or machine components. The main memory 620 and the static memory 630 are computer-readable mediums from which data and executable software instructions can be read by a computer (e.g., the processor 610). Each of the main memory 620 and the static memory 630 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 600 further includes a video display unit 650, such as a liquid crystal display (LCD), an organic light emitting diode (OLED), a flat panel display, a solid-state display, or a cathode ray tube (CRT), for example. Additionally, the computer system 600 includes an input device 660, such as a keyboard/virtual keyboard or touch-sensitive input screen or speech input with speech recognition, and a cursor control device 670, such as a mouse or touch-sensitive input screen or pad. The computer system 600 also optionally includes a disk drive unit 680, a signal generation device 690, such as a speaker or remote control, and/or a network interface device 640.

In an embodiment, as depicted in FIG. 6, the disk drive unit 680 includes a computer-readable medium 682 in which one or more sets of software instructions 684 (software) are embedded. The sets of software instructions 684 are read from the computer-readable medium 682 to be executed by the processor 610. Further, the software instructions 684, when executed by the processor 610, perform one or more steps of the methods and processes as described herein. In an embodiment, the software instructions 684 reside all or in part within the main memory 620, the static memory 630 and/or the processor 610 during execution by the computer system 600. Further, the computer-readable medium 682 may include software instructions 684 or receive and execute software instructions 684 responsive to a propagated signal, so that a device connected to a network 601 communicates voice, video or data over the network 601. The software instructions 684 may be transmitted or received over the network 601 via the network interface device 640.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, late-onset neonatal sepsis predictions provides for a clinically acceptable and validatable risk score for late-onset neonatal sepsis. The predictions may be used to reduce excessive use of antibiotics and avoid associated adverse outcomes. Additionally, the early prediction of late-onset neonatal predictions may provide for better outcomes, and resolves a question that has been difficult to address for neonates due, for example, to the lack of clinically accepted criteria warranting suspicion. The predictive model may help the medical facilities such as hospitals to improve resource allocation such as by increasing the accuracy of expected rates of hospital-acquired infection, utilization of antibiotics and numbers of required monitoring devices in a neonatal ward, utilization of disposables, and routine cleaning and maintenance required pre-discharge and post-discharge for units.

Optimal utilization of antibiotics has potential to improve neonatal outcomes. A tool that can predict onset of infection sooner than from clinical suspicion guides physicians in their decision to prescribe antibiotics. Further, the tool can guide physicians in decisions to not use antibiotics, thereby protecting the developing neonatal immune system from unnecessary treatment.

The solutions described herein may be run continuously using a feed of neonatal data collected from or on the way to the EMR system 170. In some embodiments, raw monitoring data may be used, and not only data entered by clinicians such as vital signs and other parameters entered at the end of shifts by nurses. Therefore, in the future, it may be advantageous to have the algorithm use raw monitoring data.

Although late-onset neonatal sepsis predictions has been described with reference to several exemplary embodiments, it is understood that the words that have been used are words of description and illustration, rather than words of limitation. Changes may be made within the purview of the appended claims, as presently stated and as amended, without departing from the scope and spirit of late-onset neonatal sepsis predictions in its aspects. Although late-onset neonatal sepsis predictions has been described with reference to particular means, materials and embodiments, late-onset neonatal sepsis predictions is not intended to be limited to the particulars disclosed: rather late-onset neonatal sepsis predictions extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A controller, comprising:
a memory that stores instructions; and
a processor that executes the instructions, wherein, when executed by the processor the instructions cause the controller to:
obtain and store measurements of vital signs of a neonatal patient and laboratory results from laboratory tests of the neonatal patient;
query for new measurements of vital signs of the neonatal patient and new laboratory results from laboratory tests of the neonatal patient and retrieve the new measurements of vital signs of the neonatal patient and the new laboratory results from laboratory tests of the neonatal patient;
append the new measurements of vital signs of the neonatal patient and the new laboratory results from laboratory tests of the neonatal patient to previous measurements of vital signs of the neonatal patient and previous laboratory results from laboratory tests of the neonatal patient;
apply trained artificial intelligence to the new measurements of vital signs of the neonatal patient and the new laboratory results from laboratory tests of the neonatal patient, to changes between the new measurements of vital signs of the neonatal patient and the previous measurements of vital signs of the neonatal patient, and to changes between the new laboratory results from laboratory tests of the neonatal patient and the previous laboratory results from laboratory tests of the neonatal patient, and
compute, based on applying the trained artificial intelligence after retrieving the new measurements of vital signs of the neonatal patient and the new laboratory results from laboratory tests of the neonatal patient, a determination in advance indicating whether the neonatal patient will be diagnosable with late-onset neonatal sepsis.

2. The controller of claim 1, wherein the instructions further cause the controller to:
repeatedly querying for new measurements; and
apply the trained artificial intelligence each time a query identifies a new measurement of vital signs or a new laboratory result from a laboratory test of the neonatal patient, until reaching a determination indicating that the neonatal patient will likely be infected with late-onset sepsis.

3. The controller of claim 1, wherein the instructions further cause the controller to:
compute the determination indicating whether the neonatal patient will be diagnosable with late-onset sepsis in a future period from six hours to twenty four hours in advance.

4. The controller of claim 1, wherein the instructions further cause the controller to:
periodically query for new measurements; and
apply the trained artificial intelligence each time a query identifies a new measurement of vital signs or a new laboratory result, until reaching a determination indicating that the neonatal patient will likely be infected with late-onset sepsis.

5. The controller of claim 1, wherein the instructions further cause the controller to:
identify a relative importance of each new measurement in computing the determination in advance indicating whether the neonatal patient will be diagnosable with late-onset sepsis.

6. A method, comprising:
obtaining and storing measurements of vital signs of a neonatal patient and laboratory results from laboratory tests of the neonatal patient;
querying for new measurements of vital signs of the neonatal patient and new laboratory results from laboratory tests of the neonatal patient and retrieving the new measurements of vital signs of the neonatal patient and the new laboratory results from laboratory tests of the neonatal patient;
appending the new measurements of vital signs of the neonatal patient and the new laboratory results from laboratory tests of the neonatal patient to previous measurements of vital signs of the neonatal patient and previous laboratory results from laboratory tests of the neonatal patient;
applying trained artificial intelligence to the new measurements of vital signs of the neonatal patient and the new laboratory results from laboratory tests of the neonatal patient, to changes between the new measurements of vital signs of the neonatal patient and the previous measurements of vital signs of the neonatal patient, and to changes between the new laboratory results from laboratory tests of the neonatal patient and the previous laboratory results from laboratory tests of the neonatal patient, and
computing, based on applying the trained artificial intelligence after retrieving the new measurements of vital signs of the neonatal patient and the new laboratory results from laboratory tests of the neonatal patient, a determination in advance indicating whether the neonatal patient will likely be diagnosable with late-onset neonatal sepsis.

7. The method of claim 6, wherein the trained artificial intelligence is applied by:
generating a feature vector for each new measurement of vital signs or new laboratory result from a laboratory test of the neonatal patient; and
applying each feature vector to the trained artificial intelligence.

8. The method of claim 6, wherein the trained artificial intelligence is applied by:
generating a feature vector for a time-series characteristic based on each new measurement of vital signs or new laboratory result from a laboratory test of the neonatal patient; and
applying the feature vector to the trained artificial intelligence.

9. The method of claim 6, wherein the measurements of vital signs of the neonatal patient and laboratory results from laboratory tests of the neonatal patient are obtained and stored by monitoring transmissions sent to an electronic medical record database, recording the measurements of vital signs of the neonatal patient and laboratory results from laboratory tests of the neonatal patient in the transmissions, and storing the measurements of vital signs of the neonatal patient and laboratory results from laboratory tests of the neonatal patient in a dedicated database.

10. The method of claim 6, further comprising:
training the trained artificial intelligence by correlating measurements of vital signs of a plurality of neonatal patients and laboratory results from laboratory tests of the plurality of neonatal patients with states of the plurality of neonatal patients.

11. A system, comprising:
a memory that stores instructions; and
a processor that executes the instructions, wherein, when executed by the processor the instructions cause the system to:
obtain and store measurements of vital signs of a neonatal patient and laboratory results from laboratory tests of the neonatal patient;
query for new measurements of vital signs of the neonatal patient and new laboratory results from laboratory tests of the neonatal patient and retrieve the new measurements of vital signs of the neonatal patient and the new laboratory results from laboratory tests of the neonatal patient;
append the new measurements of vital signs of the neonatal patient and the new laboratory results from laboratory tests of the neonatal patient to previous measurements of vital signs of the neonatal patient and previous laboratory results from laboratory tests of the neonatal patient;
apply trained artificial intelligence to the new measurements of vital signs of the neonatal patient and the new laboratory results from laboratory tests of the neonatal patient, to changes between the new measurements of vital signs of the neonatal patient and the previous measurements of vital signs of the neonatal patient, and to changes between the new laboratory results from laboratory tests of the neonatal patient and the previous laboratory results from laboratory tests of the neonatal patient, and
compute, based on applying the trained artificial intelligence after retrieving the new measurements of vital signs of the neonatal patient and the new laboratory results from laboratory tests of the neonatal patient, a determination in advance indicating whether the neonatal patient will be diagnosable with late-onset neonatal sepsis.

12. The system of claim 11, further comprising:
a dedicated database that stores the measurements of vital signs of the neonatal patient and laboratory results from laboratory tests of the neonatal patient.

13. The system of claim 12, further comprising:
a monitor that monitors transmissions sent to an electronic medical record database, that intercepts the measurements of vital signs of the neonatal patient and laboratory results from laboratory tests of the neonatal patient in the transmissions, and that stores the measurements of vital signs of the neonatal patient and laboratory results from laboratory tests of the neonatal patient in the dedicated database.

14. The system of claim 12, wherein the query for new measurements comprises a query to the dedicated database.

15. The system of claim 11, further comprising:
a monitor that displays a probability of the neonatal patient being diagnosable with late-onset neonatal sepsis and a parameter used to determine in advance the probability of the neonatal patient being diagnosable with late-onset neonatal sepsis.
